(19) United States
(12) Patent Application Publication
(10) Pub. No.: US 2003/0022358 A1
(43) Pub. Date: Jan. 30, 2003

(54) Growth of filterable organisms that require non-diffusable media components

(76) Inventors: Jane H. Hall, Sunnyvale, CA (US); Kenneth Borchardt, Novato, CA (US); Robert D. Hall, Sunnyvale, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/155,799

(22) Filed: May 24, 2002

Related U.S. Application Data
(60) Provisional application No. 60/294,304, filed on May 29, 2001.

(51) Int. Cl. C12N 1/20; C12N 1/18; C12N 1/16

(52) U.S. Cl. 435/252.1; 435/254.1

(57) ABSTRACT

Provided are membrane filters and methods of trapping, and culturing microorganisms. The membrane filters trap or filter from a sample a specific microorganism of interest requiring a set of growth components. The membrane comprises a first subset of the set of growth components such that the microorganism can grow and divide with the membrane contacting a medium comprising a second subset of the set of growth components defined as all components of the set of growth components not included in the first subset. Methods for detecting a microorganism comprise: (i) providing a membrane filter, comprising a first subset of the set of growth components; (ii) filtering a sample through the filter; and (iii) placing the filter in contact with a medium comprising a second subset of the set of components for growth. For members of genus Legionella a filter impregnated with activated charcoal may be employed for detection and culturing.

### RELATED APPLICATIONS

This application claims the benefit of U.S. Ser. No. 60/294,304, filed May 29, 2001, which application is fully incorporated herein by reference.

### BACKGROUND OF THE INVENTION

1. Field of the Invention

This invention relates to generally to membranes and the growth of filterable organisms, and more particularly membrane suitable for the growth of filterable organisms that require non-diffusable media components.

2. Description of Related Art

In order to detect microbial pathogens in specimens, whether of human, animal or environmental origin, the following general procedure is commonly used: the target (and other) microbes contained by the specimen are inoculated as part of the specimen into a culture medium, which provides all required nutrients for growth.

The specimen may be an untreated natural sample, or it may be pretreated as, for example, by membrane filtration, thus concentrating or amplifying the total microbe content that can be practicably cultured in a laboratory culture setup for a total specimen of a certain size. Thus, for example a 1 liter sample of environmental water may be passed through a filter having a pore size distribution which would trap at least 85% of normally sized bacteria, e.g. excluding rickettsia, chlamydia and mycoplasma the majority of which would pass through most filters with such a pore size distribution. Such membrane filtration permits culturing of substantially all the target microbes from the sample in one petri dish rather than using 1000 petri dishes each inoculated with a 1 ml fraction of the 1 liter sample.

The culture medium has the nutrients selectively required by the target microbe in addition to commonly required nutrients to permissively facilitate growth of microbes, including the target microbe requiring the selectively required nutrients in addition to commonly required nutrients. Such permissive selection facilitates survival of the target microbe in addition to other microbes having similar selective requirements, and microbes having more minimal growth requirements. Microbes requiring nutrients not included in the medium are selected against, thereby reducing the proportion of unwanted microbes and increasing the proportion of the target microbes present that grow in the sample. Such negative selection amplifies signal to noise ratio (S/N) by selecting against microorganisms other than the one targeted.

Other negatively selective additives and conditions, including antimetabolites, antibiotics, temperature and O₂ tension, which are selectively active against microbes other than the target microbes also amplify S/N by negative selection, e.g. selection against non-targeted or contaminant microorganisms. Even with the selective chemicals, the culture medium is typically a “general medium” in that the growth of both target microbes and related microbes is supported. Thus a selective medium is typically only partially specific to the target microbes.

The culture medium may be a water solution or a water gel, which is sterilized to remove any contaminating microbes possibly present in the medium which could interfere with the analysis. The culture medium must be packaged and stored in a manner that avoids contamination by microorganisms and/or degradation after manufacture, often requiring refrigeration for storage.

After one or more of the culture media are inoculated with the specimen, the inoculated media are incubated under controlled atmospheric conditions. After incubation, the culture media are examined for growth of any microbes. Certain media contain moieties that indicate the presence of a specific microorganism by a change in an observable physical condition. Alternatively or in addition, a sample of any cultured organism may be taken for further analysis. Often a sufficiently specific indicator moiety for a target organism of interest will not be available, and the presence of the target microbe can only be established by isolating it in the pure state, rather than mixed with other microbes. Once isolated on subsequent culture media, the target microbes are identified by testing for a variety of physical and chemical characteristics. Alternatively an organism identified by an appropriate indicator moiety may be cultured for the purpose of identifying the specific strain, serotype and/or subtype by methods including surface antigenic analysis and/or partial or complete sequencing of the genome. If the apparent target microbe growths are not isolated, false negative test results can occur.

Membrane filtration methods can reduce the likelihood of false positives by permitting a larger raw sample per culture compared to methods not employing a filter, but only to the extent that the target microbe can grow and multiply as trapped in or on the membrane filter.

Microorganisms requiring certain growth factors which are not freely diffusible into and/or through the membrane filter may not grow efficiently when the membrane filter is placed on the growth mediums. In this context efficiency is defined in terms of the proportion of trapped organisms forming detectable colonies upon culture with the membrane placed in contact with a medium comprising all required growth components and factors. Lack of efficient growth of membrane filter trapped target microbes because of membrane diffusion characteristics of one or more required growth components may lead to false negatives. Thus the desirable ability of membrane filtration techniques to concentrate microorganisms to increase the likelihood of detecting a microorganism of interest can be defeated by the target microbe requiring growth components that diffuse insufficiently through the membrane filter employed to support the target microorganism's growth.

A need therefore exists for improved membrane filters and methods of use therefor in the context of membrane filtration microorganism culture and detection techniques.

### SUMMARY OF THE INVENTION

Accordingly, an object of the present invention is to provide improved membrane filters, and their methods of use, for microorganism culture and detection.

Another object of the present invention is to provide improved membrane filters, and their methods of use, that trap, and culture microorganisms for diagnostic and environmental detection and scientific study.

Yet another object of the present invention is to provide improved membrane filters, and their methods of use, that can isolate specified microorganisms from a sample.

A further object of the present invention is to provide membranes for growing microorganisms, and their methods of use, where a microorganism that is unable to traverse the membrane can grow if the membrane is interposed between the microorganism and a second subset of the set of growth components.

Another object of the present invention is to provide membranes and growing microorganism, and their methods of use, particularly directed to microorganisms that are members of genus Legionella.

### BRIEF DESCRIPTION OF FIGURES

The various features and aspects of the instant invention may be more readily understood, in conjunction with the description to follow, by reference to the following drawings:

FIG. 1 depicts a cellulose fiber membrane embodiment of the invention disposed on an agar medium.

FIG. 2 depicts a microfabricated membrane embodiment of the invention disposed on an agar medium.

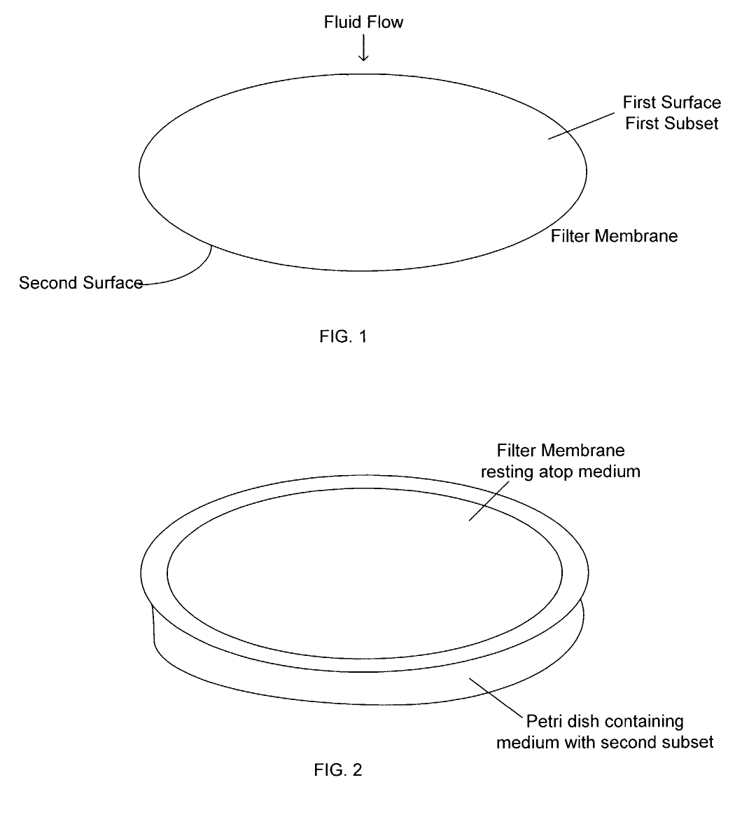

### DETAILED DESCRIPTION OF THE INVENTION

In various embodiments, the present invention is directed to membrane filters and methods of use thereof in trapping, and culturing microorganisms, for diagnostic and environmental detection and scientific study. The methods employ, and membrane filters of the invention comprise a membrane having appropriate pore size distribution for trapping or filtering out of a sample passed therethrough a specific microorganism or organism of interest requiring a set of growth components. The membrane filter has a first surface, a second surface and a designated pore size distribution whereby passing the sample through the membrane traps the specific microorganism in a proportion according to the designated pore size distribution, to the first surface of the membrane. The membrane filter of the invention has a membrane comprising a first subset of the set of growth components, on attached to, embedded in or comprising its first surface.

Typically a microorganism unable to traverse the membrane can grow if the second surface of the membrane is in contact with a medium comprising a second subset of the set of growth components. In a typical embodiment, the specific microorganism can divide on a first surface of the membrane, where the microorganism is trapped or captured by filtration, when the membrane is disposed with a second surface contacting a medium comprising a second subset of the set of components for growth. The first subset is defined as the set of all growth components that will not pass through the membrane filter. The second subset of growth components may include all components. The microorganism can grow on the membrane in contact with the medium because the first subset of required growth components are in contact with the organisms on the membrane and the second subset are provided by diffusion from the medium. The membrane and medium together provide a complete complement of the set of required growth components.

The membrane filter may be employed to detect the presence of the specific organism of interest in a sample. Specifically, a microorganism of interest having a size in a specified size distribution wherein a significant proportion of a population of the microorganism are trapped by the membrane, e.g are unable to traverse or do not pass through the membrane. Thus a specified microorganism of interest can be isolated from the sample and cultured by placing the membrane on a medium comprising a subset of the set of growth components when the additional required growth components comprise the membrane.

The membrane may comprise a membranous matrix impregnated with the first subset of the set of growth components, or to which required growth components from the first subset of the set of growth components are attached. The required growth components from the first subset of the set of growth components may be attached by chemisorption, physisorption or specific binding. Chemisorption may be, for example, by a covalent bond or an ionic bond. Physisorption, for example, by a van der Waals interaction, a hydrophobic interaction or a macromolecular electrostatic interaction. Attachment by specific binding may be, for example, by an immunoglobulin, a protein domain and a fusion protein, as for example when the membrane comprises cellulose and the attachment is by a fusion protein comprising a cellulase binding domain. Or, the membrane may comprise a membranous matrix, the membranous matrix comprising the first subset of the set of growth components.

In one embodiment the membranous matrix is impregnated with carbon. This embodiment is for detecting and culturing a microorganism that is a member of genus Legionella such as L. pneumophila, L. feelei, L. bozemanii and L. dumoffi. Such organisms require elemental carbon in addition to a medium containing yeast extract, ACES buffer, L. ketoglutarate, potassium hydroxide, L. cysteine, HCL, Ferric pyrophosphate.

Any membrane having an appropriate pore size distribution for the microorganism of interest may be employed. Fiber based membranes including cellulose and synthetic polymeric membrane filters may be employed for most prokaryotes and hence bacteria, with the exception of Chlamydia, Rickettsia and viruses. The membrane may also be microfabricated by photolithographic and other means, including the formation of sacrificial layers.

Microfabricated membranes have the advantage of greater pore size uniformity and uniformity of spatial distribution. The microfabricated membrane may be made from any material, including, for example, crystalline and glassy or amorphous materials, which may comprise molecules or atoms comprising metallic, non-metallic or semi-metallic atoms. Semimetallic materials such as the semiconductor materials, including Si and Ge and GaAs, as well as polymeric materials including polypropylene, polystyrene, polybutylene polyethylene of various densities may be employed. Polymeric materials, inorganic glasses and semiconductor materials may be microfabricated by photolithographic methods, with semiconductor materials offering the possibility of sacrificial layer formation. Formation of a sacrificial layer permits pore widths to the nanometer range, permitting capture of viruses. Mono- and poly-crystalline materials and inorganic glasses such as SiO₂ may be coated with polycrystalline C by known methods for increased strength and resistance to X-rays and the like. With crystalline substrate materials, methods of employing sacrificial layers, often crystalline permitting precise control of dimension by epitaxial growth achieved via molecular beam or chemical vapor deposition or the like. A discussion of microfabrication techniques pertinent to forming membrane and non-membrane filters and other structures, employing a Si substrate and sacrificial layers in combination with photolithography may be found in U.S. Pat. Nos. 6,044,981, 5,585,328, 5,985,164, 5,798,042 and 5,770,076 to Chu et al, U.S. Pat. Nos. 6,105,599, 5,948,255, 5,893,974 and 5,651,900 to Keller et al, and U.S. Pat. No. 5,981,923 to Tu et al, all incorporated herein by reference.

Various membrane pore size distributions will be appropriate depending upon the organism to be detected, the size of the sample and the sensitivity required for the detection assay. By varying the pore size distribution of the filter membrane, both in terms of mean pore size and statistical measure of pore size dispersion such as standard deviation or variance, membranes filtering or trapping a specified proportion of a population of specific microorganism, which will have a size distribution that is approximately known may be designed. For example the proportion of microorganisms in population in an environmental sample that are captured according to the designated pore size range distribution may be at least: 25 percent, 50 percent, 75 percent, 95 percent and 99 percent.

Typical methods of the invention for detecting a microorganism of interest requiring a set of growth components comprise: (i) providing a membrane filter, preferably one having a designated pore size distribution, the membrane filter membrane having a first subset of the set of growth components; (ii) filtering a sample through the filter; and (iii) placing the filter in contact with a medium having a second subset of the set of components for growth. The first subset is defined as above as all required growth components that will not pass through the filter membrane. For members of genus Legionella the first subset is carbon as activated charcoal. A cellulose or paper filter or man-made material which brings activated charcoal into contact with organisms may be employed according to the invention for detecting and culturing Legionella.

### EXAMPLE 1

### Legionella w. Charcoal Impregnated Millipore

[Experiments, Data from disclosure comparing colony nos.]

### EXAMPLE 2

### Acid sensitive Marble/ Limestone Impregnated

[On an agar suitable for culture but for absence of marble chips vs marble agar, having less available acid neutralizing surface]

### EXAMPLE 3

### Microfabricated Membrane for Viruses

[attach appropriate cell host via cellulose coat and fusion proteins post-filtration]

Although exemplary embodiments of the instant invention have been described and depicted, it will be apparent to the artisan of ordinary skill that a number of changes, modifications, or alterations to the invention as described herein may be made, none of which depart from the spirit of the instant invention. All such changes, modifications, and alterations should therefore be seen as within the scope of the instant invention.

While the instant invention is disclosed with reference to preferred embodiments detailed above, it is to be understood that these embodiments are intended in an illustrative or exemplary rather than in a limiting sense, as it is contemplated that modifications will readily occur to those skilled in the art, modifications which will be within the spirit of the invention and the scope of the appended claims. All patents, papers, articles, references and books cited herein are incorporated by reference in their entirety

We claim:

1. A membrane for growing a microorganism requiring a set of growth components, the membrane comprising a first subset of the set of growth components,
whereby a microorganism unable to traverse the membrane can grow if the membrane is interposed between the organism and a medium comprising a second subset of the set of growth components defined as all components of the set of growth components not included in the first subset of the set of growth components.

2. The membrane of claim 1, wherein the membrane comprises a membranous matrix impregnated with the first subset of the set of growth components.

3. The membrane of claim 1, wherein the membrane comprises a membranous matrix, the membranous matrix comprising the first subset of the set of growth components.

4. The membrane of claim 1, wherein the membrane comprises a membranous matrix to which required growth components from the first subset of the set of growth components are attached.

5. The membrane of claim 4, wherein the required growth components from the first subset of the set of growth components are attached by an attachment selected from the group consisting of chemisorption, physisorption and specific binding.

6. The membrane of claim 5, wherein the attachment is chemisorption selected from the group consisting of a covalent bond and an ionic bond.

7. The membrane of claim 5, wherein the attachment is physisorption selected from the group consisting of a van der Waals interaction, a hydrophobic interaction and a macromolecular electrostatic interaction.

8. The membrane of claim 5, wherein the attachment is specific binding selected from the group consisting of an immunoglobulin, a protein domain and a fusion protein.

9. The membrane of claim 2 or 7, wherein the membranous matrix is impregnated with carbon.

10. The membrane of claim 8, wherein the membrane comprises cellulose and the attachment is by a fusion protein comprising a cellulase binding domain.

11. The membrane of claim 1 or 9, wherein the microorganism is a member of genus Legionella.

12. The membrane of claim 11, wherein the microorganism is selected from the group consisting of L. pneumophila, L. feelei, L. bozemanii and L. dumoffi.

13. A membrane for trapping and growing a microorganism of interest requiring a set of growth components, the membrane comprising a first subset of the set of growth components,
whereby a microorganism of interest of size in a specified size distribution wherein a significant proportion of a population are trapped by the membrane can grow if the membrane is placed on a medium comprising a second subset of the set of growth components defined as all components of the set not included in the first subset.

14. A membrane filter for growing a microorganism requiring a set of components for growth, the membrane comprising a first subset of the set of components for growth,
whereby a microorganism not passing through the membrane can grow if the membrane is interposed between the organism and a medium comprising a second subset of the set of components for growth comprising the first set of components for growth not included in the first subset of the set of components for growth.

15. A membrane filter having a first surface, a second surface and a designated pore size distribution, the membrane filter for detecting a specific microorganism in a sample requiring a set of components for growth, the membrane comprising a first subset of components for growth of the specific microorganism,
whereby passing the sample through the membrane traps the specific microorganism in a proportion according to the designated pore size range distribution, to the first surface of the membrane,
wherein the specific microorganism can divide on the first surface of the membrane when the membrane is disposed with the second surface contacting a medium comprising a second subset of the set of components for growth, the second subset defined as all components of the first set not included in the first subset.

16. The membrane filter of claim 15, wherein the proportion according to the designated pore size range distribution is at least 25 percent.

17. The membrane filter of claim 15, wherein the proportion according to the designated pore size range distribution is at least 50 percent.

18. The membrane filter of claim 15, wherein the proportion according to the designated pore size range distribution is at least 75 percent.

19. The membrane filter of claim 15, wherein the proportion according to the designated pore size range distribution is at least 95 percent.

20. The membrane filter of claim 15, wherein the proportion according to the designated pore size range distribution is at least 95 percent.

21. The membrane filter of claim 15, wherein the proportion according to the designated pore size range distribution is at least 99 percent.

22. The membrane filter of claim 15, wherein the membrane filter comprises a membranous matrix impregnated with the first subset of the set of growth components.

23. The membrane filter of claim 15, wherein the membrane filter comprises a membranous matrix, the membranous matrix comprising the first subset of the set of growth components.

24. The membrane filter of claim 15, wherein the membrane filter comprises a membranous matrix to which required growth components from the first subset of the set of growth components are attached.

25. The membrane filter of claim 24, wherein the required growth components from the first subset of the set of growth components are attached by an attachment selected from the group consisting of chemisorption, physisorption and specific binding.

26. The membrane of claim 25, wherein the attachment is chemisorption selected from the group consisting of a covalent bond and an ionic bond.

27. The membrane of claim 25, wherein the attachment is physisorption selected from the group consisting of a van der Waals interaction, a hydrophobic interaction and a macromolecular electrostatic interaction.

28. The membrane of claim 25, wherein the attachment is specific binding selected from the group consisting of an immunoglobulin, a protein domain and a fusion protein.

29. The membrane filter of claim 23 or 27, wherein the membranous matrix is impregnated with carbon.

30. The membrane filter of claim 28, wherein the membrane comprises cellulose and the attachment is by a fusion protein comprising a cellulase binding domain.

31. The membrane filter of claim 22 or 29, wherein the microorganism is a member of genus Legionella.

32. The membrane filter of claim 31, wherein the microorganism is selected from the group consisting of L. pneumophila, L. feelei, L. bozemanii and L. dumoffi.

33. A method of detecting a microorganism of interest requiring a set of growth components comprising:
(i) providing a membrane filter for growing a microorganism of interest requiring a set of components for growth, the membrane comprising a first subset of the set of growth components of a microorganism of interest;
(ii) filtering a sample through the filter provided in step (i);
(iii) placing the filter in contact with a medium comprising a second subset of the set of components for growth, the second subset defined as all components of the first set not included in the first subset.

34. The method of claim 33, wherein the membrane filter provided in step (i) comprises a membranous matrix impregnated with the first subset of the set of growth components.

35. The method of claim 33, wherein the membrane filter provided in step (i) comprises a membranous matrix, the membranous matrix comprising the first subset of the set of growth components.

36. The method of claim 33, wherein the membrane filter provided in step (i) comprises a membranous matrix to which required growth components from the first subset of the set of growth components are attached.

37. The method of claim 37, wherein the membrane filter provided in step (i) comprises the required growth components of the first subset of the set of growth components attached by an attachment selected from the group consisting of chemisorption, physisorption and specific binding.

38. The method of claim 37, wherein the attachment is chemisorption selected from the group consisting of a covalent bond and an ionic bond.

39. The method of claim 37, wherein the attachment is physisorption selected from the group consisting of a van der Waals interaction, a hydrophobic interaction and a macromolecular electrostatic interaction.

40. The method of claim 37, wherein the attachment is specific binding selected from the group consisting of an immunoglobulin, a protein domain and a fusion protein.

41. The method of claim 34 or 39, wherein the membranous matrix is impregnated with carbon.

42. The method of claim 41, wherein the membrane filter provided in step (i) comprises cellulose and the attachment is by a fusion protein comprising a cellulase binding domain.

43. The method of claim 33, wherein the microorganism of interest is a member of genus Legionella.

44. The method of claim 33, wherein the microorganism of interest is selected from the group consisting of L. pneumophila, L. feelei, L. bozemanii and L. durmoffi.

45. A method of detecting a specific microorganism requiring a set of growth components comprising:
(i) providing a membrane filter having a designated pore size distribution, the membrane filter, the membrane comprising a first subset of the set of growth components;
(ii) filtering a sample through the filter provided in step (i) to trap the specific microorganism in a proportion according to the designated pore size range distribution;
(iii) placing the filter in contact with a medium comprising a second subset of the set of components for growth, the second subset defined as all components of the first set not included in the first subset.

46. The method of claim 45, wherein the proportion according to the designated pore size range distribution of step (ii) is at least 25 percent.

47. The method of claim 45, wherein the proportion according to the designated pore size range distribution of step (ii) is at least 50 percent.

48. The method of claim 45, wherein the proportion according to the designated pore size range distribution of step (ii) is at least 75 percent.

49. The method of claim 45, wherein the proportion according to the designated pore size range distribution of step (ii) is at least 95 percent.

50. The method of claim 45, wherein the proportion according to the designated pore size range distribution of step (ii) is at least 95 percent.

51. The method of claim 45, wherein the proportion according to the designated pore size range distribution of step (ii) is at least 99 percent.

52. The method of claim 45, wherein the membrane filter of step (i) comprises a membranous matrix impregnated with the first subset of the set of growth components.

53. The method of claim 45, wherein the membrane filter of step (i) comprises a membranous matrix, the membranous matrix comprising the first subset of the set of growth components.

54. The method of claim 45, wherein the membrane filter of step (i) comprises a membranous matrix to which required growth components from the first subset of the set of growth components are attached.

55. The method of claim 54, wherein the membrane filter provided in step (i) comprises the required growth components of the first subset of the set of growth components attached by an attachment selected from the group consisting of chemisorption, physisorption and specific binding.

56. The method of claim 55, wherein the attachment is chemisorption selected from the group consisting of a covalent bond and an ionic bond.

57. The method of claim 55, wherein the attachment is physisorption selected from the group consisting of a van der Waals interaction, a hydrophobic interaction and a macromolecular electrostatic interaction.

58. The method of claim 55, wherein the attachment is specific binding selected from the group consisting of an immunoglobulin, a protein domain and a fusion protein.

59. The method of claim 52 or 57, wherein the membranous matrix is impregnated with carbon.

60. The method of claim 58, wherein the membrane filter provided in step (i) comprises cellulose and the attachment is by a fusion protein comprising a cellulase binding domain.

61. The method of claim 52 or 59, wherein the microorganism is a member of genus Legionella.

62. The method of claim 61, wherein the microorganism is selected from the group consisting of L. pneumophila, L. feelei, L. bozemanii and L. dumoffi.
